(19) United States
(12) Patent Application Publication
(10) Pub. No.: US 2022/0074880 A1
(43) Pub. Date: Mar. 10, 2022

(54) MEMS HYDROGEN SENSOR AND HYDROGEN SENSING SYSTEM

(71) Applicants: Hyundai Motor Company, Seoul (KR); Kia Corporation, Seoul (KR)

(72) Inventors: Dong Gu Kim, Hwaseong-si (KR); Hyun Soo Kim, Yongin-si (KR); Il Seon Yoo, Hwaseong-si (KR)

(21) Appl. No.: 17/318,678

(22) Filed: May 12, 2021

(30) Foreign Application Priority Data
Sep. 7, 2020 (KR) 10-2020-0114037

(51) Int. Cl. G01N 27/12; G01N 33/00

(52) U.S. Cl. CPC G01N 27/128; G01N 33/005

(57) ABSTRACT

Embodiments of the present invention relate to a MEMS hydrogen sensor and a system including the same. An exemplary embodiment of the present invention provides a MEMS (micro electro-mechanical systems) hydrogen sensor including a sensing element configured to sense hydrogen gas, an anti-icing element configured to surround the sensing element, and a compensation element configured to have same resistance as that of the sensing element.

### CROSS-REFERENCE TO RELATED APPLICATIONS

This application claims priority to Korean Patent Application No. 10-2020-0114037, filed on Sep. 7, 2020, which application is hereby incorporated herein by reference.

### TECHNICAL FIELD

Embodiments of the present invention generally relate to a MEMS (micro electro- mechanical systems) hydrogen sensor and a hydrogen system.

### BACKGROUND

A hydrogen sensor is an essential sensor for safety management not only in hydrogen electric vehicles but also in all areas of hydrogen production/transport/utilization. A monitoring system and a sensor for sensing hydrogen leakage are installed and operated at a place where hydrogen storages and fuel cell systems are operated.

Hydrogen is known to ignite and explode when it encounters a spark with a hydrogen gas of a concentration of 4% or more in the air and a spark of 20 uJ or more or an object with a surface temperature of 135° C. or more. As such, hydrogen has difficulty in safety and handling, so a sensor for sensing hydrogen leakage has been developed and is being applied.

In a hydrogen electric vehicle, a hydrogen sensor is installed in a storage container, near joints of a piping system, and around a fuel cell stack, and transmits a sensed hydrogen concentration value to a vehicle control system so that each control system immediately takes a measure for ensuring vehicle safety.

A hydrogen sensing technique of the hydrogen sensor is divided into catalyst, heat conduction, electrochemistry, resistance, work function, mechanical, optical, and acoustic types, and for a leakage sensor for a hydrogen electric vehicle and a hydrogen system, catalyst, heat conduction, resistance, and mechanical hydrogen sensing techniques are suitable in consideration of measured concentration/reaction rate/durability.

Among the catalytic types, the catalytic combustion hydrogen sensor measures the resistance of the heater by using the heat generated when hydrogen gas contacts the catalyst and reacts with oxygen, and an application of a MEMS structure shows a fast reaction rate and high gas selectivity, so it is currently applied to vehicles.

However, in the catalytic combustion hydrogen sensor, according to a reaction principle, reaction moisture may be generated, and thus freezing may occur on a surface of a sensing device in a harsh vehicle environment (−40° C. to 105° C.), particularly at low temperatures. In order to solve this disadvantage, an additional heater is provided to remove such freezing, and a Wheatstone bridge circuit is configured by using with a sensing element and a compensation element, to measure hydrogen concentration for temperature compensation.

As illustrated in FIG. 1A and FIG. 1B, in a conventional catalytic combustion hydrogen sensor, a total of four elements constituting each of a sensing element 40 and a compensation element 50 including external resistors R1 and R2 are included, so compensation is difficult due to high chip area consumption and low resistance difference between the elements. In addition, each element 40 has four terminals, so there are eight terminals for the two elements 40 and 50, requiring eight wire bondings, and not only a measuring circuit 20 but also a heater driving circuit 30 requiring large area consumption.

The above information disclosed in this Background section is only for enhancement of understanding of the background of the invention, and therefore, it may contain information that does not form the prior art that is already known in this country to a person of ordinary skill in the art.

### SUMMARY

Embodiments of the present invention generally relate to a MEMS (micro electro- mechanical systems) hydrogen sensor and a system including the same. Particular embodiments relate to a catalytic combustion MEMS hydrogen sensor in which a sensing element and a compensation element are integrated.

An exemplary embodiment of the present invention has been made in an effort to provide a single MEMS hydrogen sensor and a system including the same, capable of reducing a cost and minimizing a difference in resistance between elements by integrating an anti-icing function, a sensing function, and a compensation function.

The technical objects of embodiments of the present invention are not limited to the objects mentioned above, and other technical objects not mentioned can be clearly understood by those skilled in the art from the description of the claims.

An exemplary embodiment of the present invention provides a MEMS (micro electro-mechanical systems) hydrogen sensor including: a sensing element configured to sense hydrogen gas; an anti-icing element configured to surround the sensing element; and a compensation element configured to have same resistance as that of the sensing element.

In an exemplary embodiment, the MEMS hydrogen sensor may further include a catalyst layer formed at an upper portion of the sensing element to react with the hydrogen gas.

In an exemplary embodiment, the sensing element may be formed in a center of the MEMS hydrogen sensor, and the compensation element may include formed in a first direction of the sensing element.

In an exemplary embodiment, the anti-icing element may include a first anti-icing element formed at opposite sides of the sensing element in a second direction crossing the first direction; and a second anti-icing element.

In an exemplary embodiment, the MEMS hydrogen sensor may further include a plurality of electrode pads respectively provided at ends of the sensing element, the compensation element, the first anti-icing element, and the second anti-icing element.

In an exemplary embodiment, it may be formed as a single element including all of the sensing element, the compensation element, the first anti-icing element, and the second anti-icing element of a Wheatstone bridge circuit.

An exemplary embodiment of the present invention provides a MEMS hydrogen sensor including: a first sensing element configured to sense hydrogen gas; a second sensing element configured to sense the hydrogen gas; and a first and second anti-icing elements configured to surround the first sensing element and the second sensing element.

In an exemplary embodiment, the first anti-icing element may be positioned at a left side of the first sensing element and the second sensing element, and the second anti-icing element may be positioned at a right side of the first sensing element and the second sensing element.

In an exemplary embodiment, the MEMS hydrogen sensor may further include a catalyst layer formed above the first sensing element and the second sensing element.

In an exemplary embodiment, resistance values of the first sensing element and the first anti-icing element may be the same, and resistance values of the second sensing element and the second anti-icing element may be the same.

In an exemplary embodiment, when hydrogen is sensed, resistance values of the first and second anti-icing elements increase.

An exemplary embodiment of the present invention provides a MEMS hydrogen sensing system including: a MEMS hydrogen sensor configured to include a first sensing element configured to sense hydrogen gas; a second sensing element configured to sense the hydrogen gas; and a first and second anti-icing elements for surrounding the first sensing element and the second sensing element, an temperature sensor configured to sense an external temperature; and a measurement circuit configured to compensate an output signal by the hydrogen sensor by using a temperature sensing value by the temperature sensor.

The present technique may provide a single MEMS hydrogen sensor integrating an anti-icing function, a sensing function, and a compensation function to reduce a cost and minimize a difference in resistance between elements.

In addition, various effects that can be directly or indirectly identified through this document may be provided.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1A and FIG. 1B illustrate views for describing a conventional MEMS hydrogen sensor.

FIG. 2A illustrates a schematic view of a MEMS hydrogen sensor according to an exemplary embodiment of the present invention.

FIG. 2B illustrates a circuit diagram of a Wheatstone bridge according to an exemplary embodiment of the present invention.

FIG. 3 illustrates a schematic view showing a configuration of a measurement system of a MEMS hydrogen sensor according to an exemplary embodiment of the present invention.

FIG. 4 illustrates a detailed top plan view of a MEMS hydrogen sensor according exemplary embodiment of the present invention.

FIG. 5 illustrates a top plan view for comparing a sensing area of a MEMS hydrogen sensor according exemplary embodiment of the present invention.

FIG. 6A to FIG. 6K illustrate a manufacturing method of a MEMS hydrogen sensor according to an exemplary embodiment of the present invention.

FIG. 7 illustrates a configuration view of a MEMS hydrogen sensor according to another embodiment of the present invention.

FIG. 8A illustrates a view for describing a circuit configuration for hydrogen measurement of a MEMS hydrogen sensor according to another embodiment of the present invention.

FIG. 8B illustrates a flowchart for describing a sensing area of a MEMS hydrogen sensor according to another exemplary embodiment of the present invention.

FIG. 9A to FIG. 9C illustrate views for describing an effect of a MEMS hydrogen sensor according to another exemplary embodiment of the present invention.

### DETAILED DESCRIPTION OF ILLUSTRATIVE EMBODIMENTS

Hereinafter, some exemplary embodiments of the present invention will be described in detail with reference to exemplary drawings. It should be noted that in adding reference numerals to constituent elements of each drawing, the same constituent elements have the same reference numerals as possible even though they are indicated on different drawings. In addition, in describing exemplary embodiments of the present invention, when it is determined that detailed descriptions of related well-known configurations or functions interfere with understanding of the exemplary embodiments of the present invention, the detailed descriptions thereof will be omitted.

In describing constituent elements according to an exemplary embodiment of the present invention, terms such as first, second, A, B, (a), and (b) may be used. These terms are only for distinguishing the constituent elements from other constituent elements, and the nature, sequences, or orders of the constituent elements are not limited by the terms. In addition, all terms used herein including technical scientific terms have the same meanings as those which are generally understood by those skilled in the technical field to which the present invention pertains (those skilled in the art) unless they are differently defined. Terms defined in a generally used dictionary shall be construed to have meanings matching those in the context of a related art, and shall not be construed to have idealized or excessively formal meanings unless they are clearly defined in the present specification.

A MEMS (micro electro-mechanical systems) sensor is used as a tool for monitoring, detecting, and monitoring of an external environment through physical, chemical, and biological sensing by using an ultra-compact high-sensitivity sensor. Embodiments of the present invention disclosures the MEMS hydrogen sensor, and particularly disclosures a catalytic combustion hydrogen sensor.

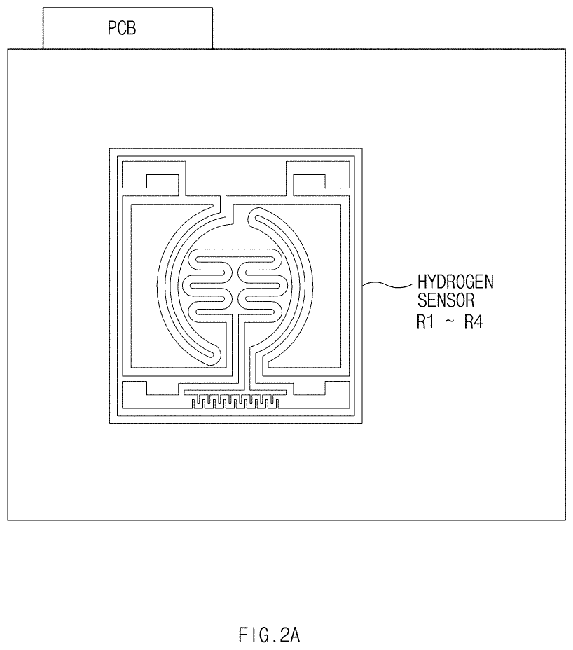

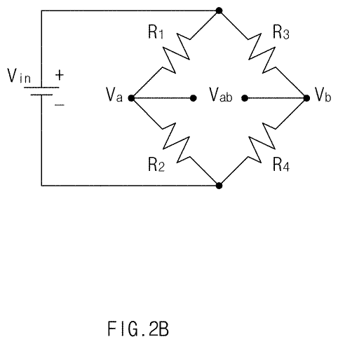

Hereinafter, exemplary embodiments of the present invention will be described in detail with reference to FIG. 2A to FIG. 9C.

FIG. 2A illustrates a schematic view of a MEMS hydrogen sensor according to an exemplary embodiment of the present invention.

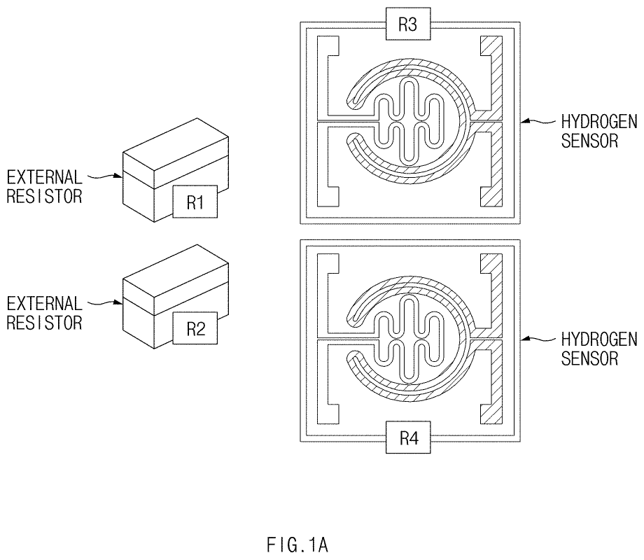

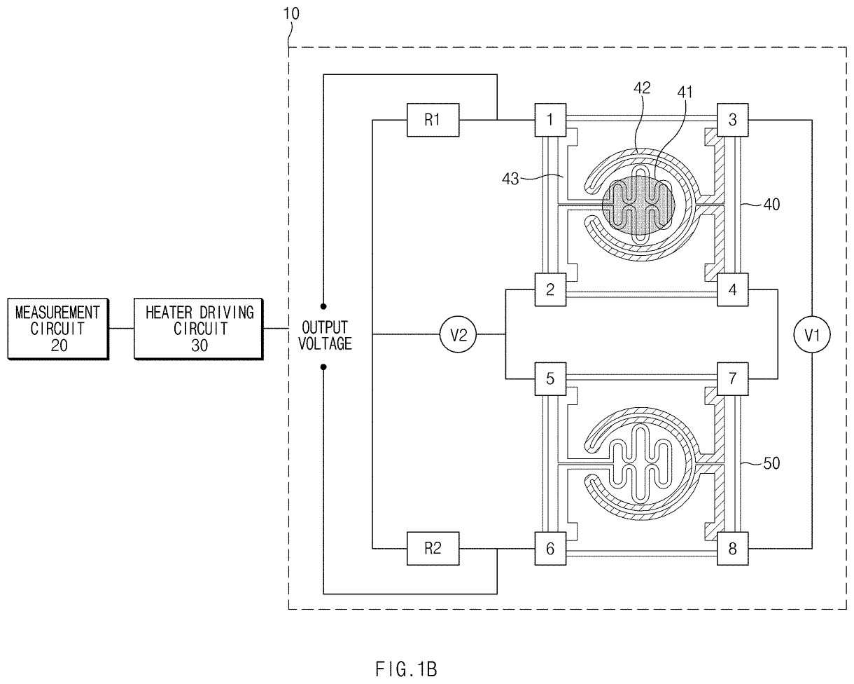

As illustrated in FIG. 1A, two external resistors and two hydrogen sensors were each conventionally independently configured on one chip, and as illustrated in FIG. 2A, the MEMS hydrogen sensor is formed to include four elements as one single element according to an exemplary embodiment of the present invention. That is, according to the present exemplary embodiment, the MEMS hydrogen sensor may reduce chip area consumption by integrating a sensing element, a compensation element, and an anti-icing element into one single element, and may reduce a cost by integrating a sensing function, a compensation function, and an ice removal function through a change of an electrode pattern shape without an additional process for single device fabrication. In addition, according to an exemplary embodiment of the present invention, the MEMS hydrogen sensor may minimize a change caused by a resistance difference between elements by preventing occurrence of the resistance difference.

FIG. 2B illustrates a circuit diagram of a Wheatstone bridge according to an exemplary embodiment of the present invention. The MEMS hydrogen sensor constitutes the Wheatstone bridge by using a sensing element with an oxidation catalyst applied to a metal wire coil and a compensation element with no oxidation catalyst applied thereto. In FIG. 2B, resistors R1, R2, R3, and R4 may be configured as a single element as illustrated in FIG. 2A.

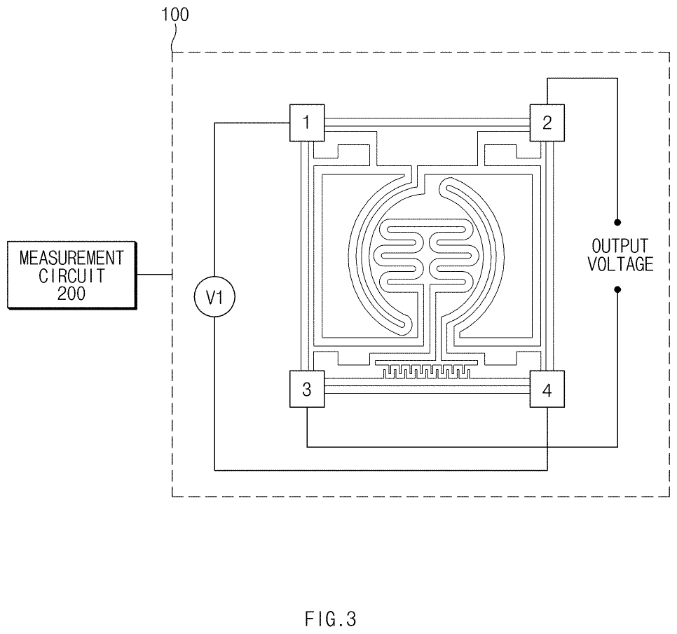

FIG. 3 illustrates a schematic view showing a configuration of a measurement system of a MEMS hydrogen sensor 100 according to an exemplary embodiment of the present invention. The measurement circuit 200 is connected to terminals 1, 2, 3, and 4 of the MEMS hydrogen sensor 100 as a single element, and a voltage is applied between the first terminal 1 and the fourth terminal 4 and an output voltage is outputted between the second terminal 2 and the third terminal 3. A change in resistance of the sensing device depending on a change in a hydrogen concentration may be measured by a change in an output voltage of a Wheatstone bridge circuit in the device.

The measurement circuit 200 may measure an output voltage of the MEMS hydrogen sensor 100 to determine whether there is a hydrogen leak. The measurement circuit 200 may be electrically connected to the hydrogen sensor 100 and may be an electric circuit that executes a command of software, thereby, performing various data processing and calculations described later. The measurement circuit 200 may be, e.g., a central processing unit (CPU), an electronic control unit (ECU), a micro controller unit (MCU), or other subcontrollers mounted in the vehicle.

According to the present exemplary embodiment, the measurement circuit 200 which is operated as the above may be implemented in a form of an independent hardware device including a memory and a processor that processes each operation, and may be driven in a form included in other hardware devices such as a microprocessor or a general purpose computer system.

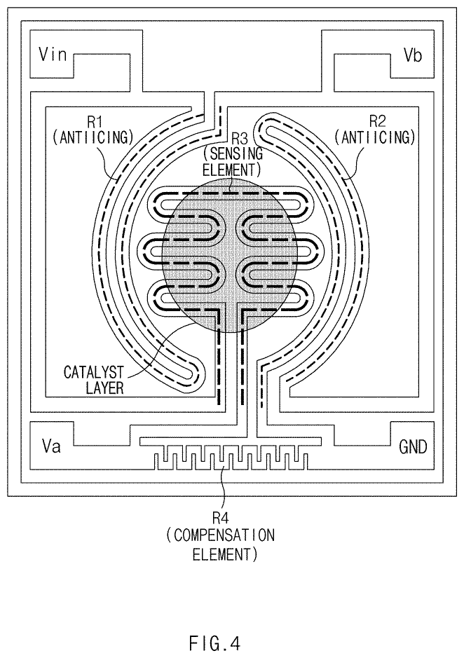

FIG. 4 illustrates a detailed configuration view of a MEMS hydrogen sensor according to an embodiment of the present invention.

The MEMS hydrogen sensor 100 is formed to include anti-icing devices R1 and R2, a sensing device R3, and a compensation device R4, and includes electrode pads each of which has an end that is connected to a voltage input terminal Vin, output voltages Va and Vb, and a ground voltage terminal GND. That is, the electrode pads are symmetrically respectively provided on outer peripheries of the MEMS hydrogen sensor, to perform electrical connection such that a voltage is applied to the MEMS hydrogen sensor. That is, each of the four resistance elements R1, R2, R3, and R4 for constituting the Wheatstone bridge circuit of FIG. 2B may be separated by using functions such as anti-icing, sensing and compensation, to perform a function for preventing low-temperature freezing of the catalytic combustion hydrogen sensor.

In this case, the sensing element R3 is disposed in a center of the MEMS hydrogen sensor, the compensation element R4 is disposed in a first direction (e.g., lower) of the sensing device, and the anti-icing elements R1 and R2 are formed at opposite sides of the sensing element R3 in a second direction (e.g., left and right), which is a direction crossing the first direction.

TABLE 1
Example of hydrogen sensing and temperature compensation using single chip

| Temp. | Hydrogen | R1 | R2 | R3 | R4 | Vab | Resistance value |
|---|---|---|---|---|---|---|---|
| Room | Off | 90Ω | 90Ω | 120Ω | 120Ω | oV | — |
| temp. | | | | | | | |
| External | Off | 100Ω | 100Ω | 140Ω | 140Ω | oV | R1 to R4 increase by |
| temp | | | | | | | increase in external |
| increase | | | | | | | temperature |
| Room | On | 90Ω | 90Ω | 130Ω | 120Ω | 0.02*Vab | R3 increase by |
| temp. | | | | | | | hydrogen reaction |
| External | On | 100Ω | 100Ω | 152Ω | 140Ω | 0.02*Vab | Increase in external |
| temp. | | | | | | | temp.: R1 to R4 |
| increase | | | | | | | Hydrogen reaction: R3 |
| | | | | | | | increase |

Table 1 shows examples of hydrogen sensing and compensation using a single element.

Referring to Table 1, it can be seen that resistance values of the respective resistance elements R1, R2, R3, and R4 all increase when an external temperature increases.

It can be seen that a resistance value of R3 increases due to a hydrogen reaction when hydrogen is on at room temperature.

It can be seen that when the external temperature increases and hydrogen is in an ON state, each of the resistance elements R1, R2, R3, and R4 increases, and the resistance value of R3 further increases by the hydrogen reaction.

According to an exemplary embodiment of the present invention, in the MEMS hydrogen sensor, the elements R1 and R2 are formed to have a circular shape at left and right sides in a form surrounding the sensing element R3 for sensing hydrogen, and the compensation element R4 having a same resistance value as that of the sensing element R3 is formed at a lower portion of the sensing element R3.

As in the Wheatstone bridge circuit of FIG. 2B, when resistance values of the resistance elements R1 and R3 are the same and the resistance values of the resistance elements R2 and R4 are the same, no voltage difference occurs, and thus Vab=0. Thereafter, when hydrogen is sensed in the sensing element R3, a reaction heat is generated by a reaction of hydrogen gas in a catalyst layer, so that the resistance value of the resistance element R2 increases, resulting in a voltage difference between output voltages Va and Vb. Accordingly, the measurement circuit 200 measures the voltage difference to determine whether hydrogen leaks.

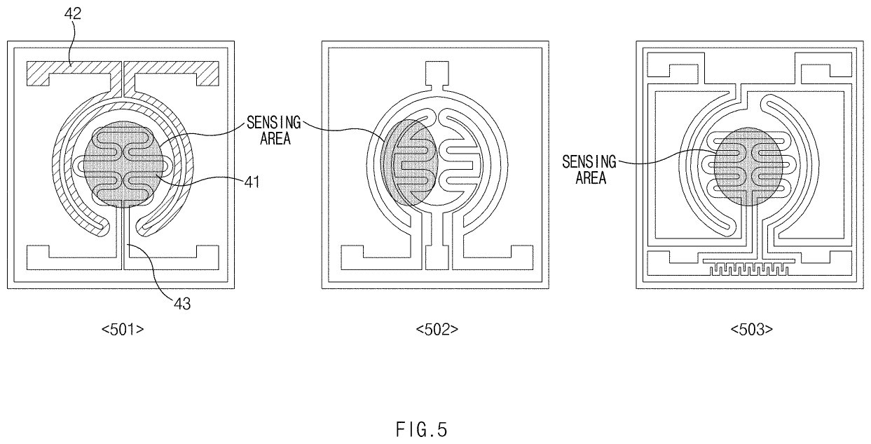

FIG. 5 illustrates a top plan view for comparing a sensing area of a MEMS hydrogen sensor according exemplary embodiment of the present invention.

Referring to a view 501 of FIG. 5, a conventional sensing element includes a catalyst layer 41, an anti-icing heater 42, and a catalytically active heater 43, and requires a compensating element including the anti-icing heater 42 and the catalytically active heater 43 without the catalyst layer.

A view 502 shows that, according to a structure in which the sensing element and the compensation element are simply integrated, a first side is driven as the sensing element and a second side is driven as the compensation element, and thus a sensing area may be narrowed by performing a sensing function only at, e.g., a left portion thereof corresponding to the sensing element.

A view 503 shows the MEMS hydrogen sensor according to an exemplary embodiment of the present invention, and it can be seen that the sensing area of the sensing element is as wide as before. That is, in the exemplary embodiment of the present invention, even when the sensing element and the compensation element are integrated, a large sensing area may be secured as before.

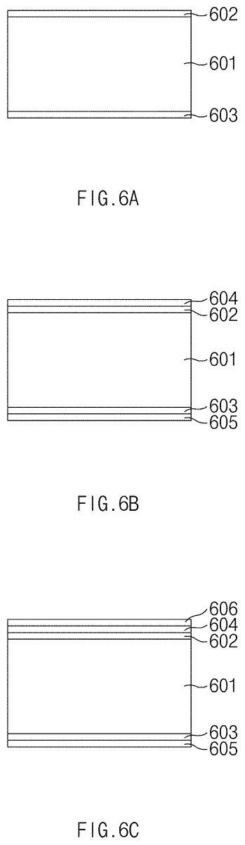

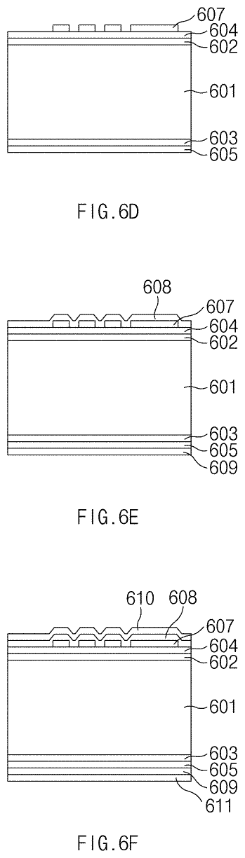

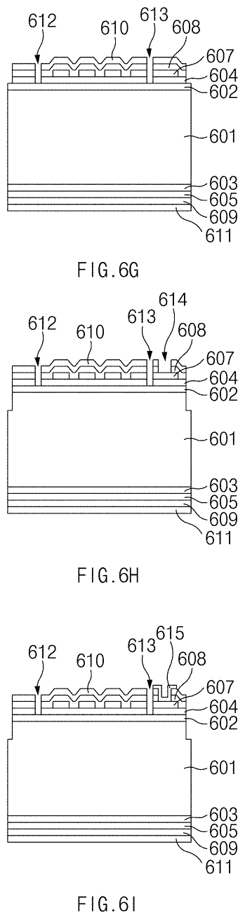

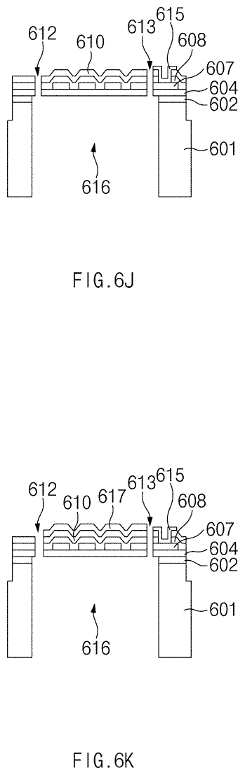

Hereinafter, a sensor manufacturing method of a MEMS hydrogen sensor according to an exemplary embodiment of the present invention will be described in detail with reference to FIG. 6A to FIG. 6K. FIG. 6A to FIG. 6K illustrates a manufacturing process of a MEMS hydrogen sensor according to an exemplary embodiment of the present invention.

First, as illustrated in FIG. 6A, first silicon oxide (SiO₂) films 602 and 603 are formed on upper and lower surfaces of a silicon (Si) substrate 601 to have a predetermined thickness by using a dry oxidation method in a state of having a thickness in a predetermined range through a back side polishing process.

Subsequently, as illustrated in FIG. 6B, first silicon nitride (Si₃N₄) films 604 and 605 are formed on an upper portion of the first silicon oxide film 602 formed on the upper surface of the silicon substrate 601 and a lower portion of the first silicon oxide film 603 formed on the lower surface of the silicon substrate 601 to have a predetermined thickness.

Next, as illustrated in FIG. 6C, a metal material for forming an electrode layer 606 is deposited on the silicon first nitride (Si₃N₄) film 604 above the silicon substrate 601. In this case, the metal material may be molybdenum.

Subsequently, as illustrated in FIG. 6D, the electrode layer 606 may be patterned to have a same pattern as the top plan view of FIG. 4.

Subsequently, as illustrated in FIG. 6E, a second silicon oxide film 608 is formed on the patterned electrode layer 607, and a second silicon oxide film 609 is formed under the first silicon nitride (Si₃N₄) film 605 and the first silicon oxide film 603 formed on the lower surface of the silicon substrate 601 to have a predetermined thickness.

Next, as illustrated in FIG. 6F, second silicon nitride (Si₃N₄) films 610 and 611 are formed at an upper portion of the second silicon oxide film 608 and at a lower portion of the second silicon oxide film 609 to have a predetermined thickness.

Thereafter, as illustrated in FIG. 6G, patterning for forming a membrane structure is performed through back etching later. That is, holes 612 and 613 are formed by etching opposite ends of a portion where a membrane structure is to be formed in a structure above the silicon substrate 601.

Subsequently, as illustrated FIG. 6H, a portion of the electrode layer 607 is exposed by performing an etching process for forming an electrode pad on the structure above the silicon substrate 601, so as to form a hole 614.

As illustrated in FIG. 6I, the electrode pad 615 is formed by depositing a metal material to a predetermined thickness in the hole 614 for forming the electrode pad.

As illustrated in FIG. 6I, the silicon substrate 601, the first silicon oxide film 602 on the upper surface of the silicon substrate 601, and the structures 602, 605, 609, 611 on the lower surface of the silicon substrate 601 are etched by using a dry method, so as to form a membrane 616.

As illustrated in FIG. 6H, a catalyst layer 617 is formed by depositing platinum (Pt) for a catalyst role at an upper portion of the second silicon nitride (Si₃N₄) film 610.

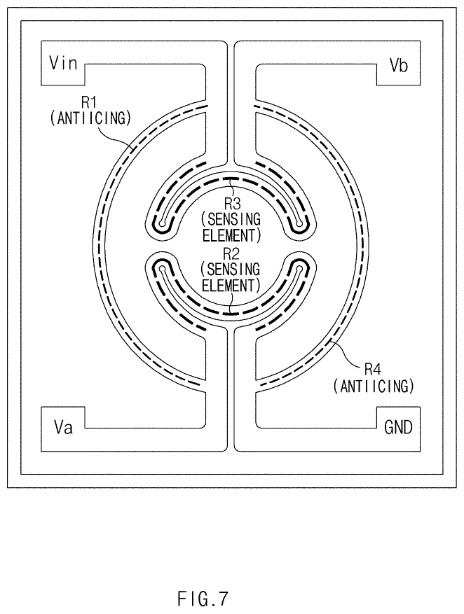

FIG. 7 illustrates a configuration view of a MEMS hydrogen sensor according to another embodiment of the present invention.

Referring to FIG. 7, the MEMS hydrogen sensor according to another exemplary embodiment of the present invention may include two anti-icing elements R1. and R4 and two sensing elements R2 and R3 instead of the compensation element. In this case, resistance values of the anti-icing element R1 and the sensing element R3 are the same, and resistance values of the anti-icing element R4 and the sensing element R2 are the same. Thereafter, when a reaction heat is generated by hydrogen gas, the resistance values of the anti-icing elements R1 and R4 increase, so that the output voltage Vab increases twice.

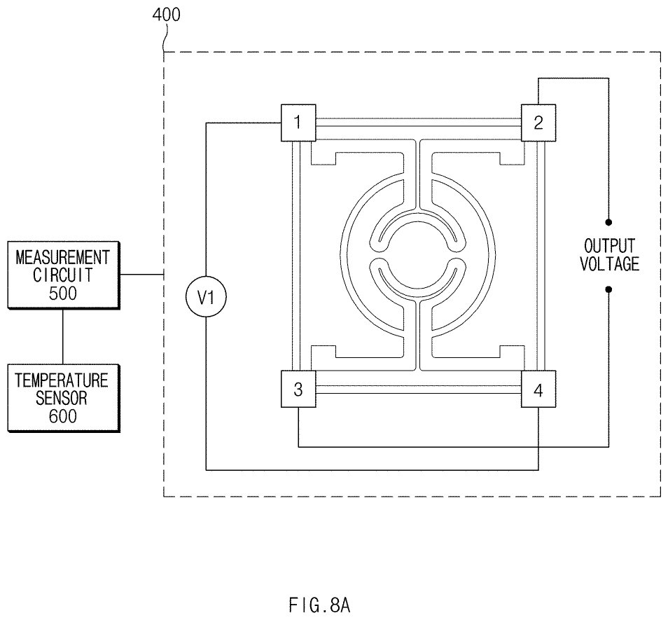

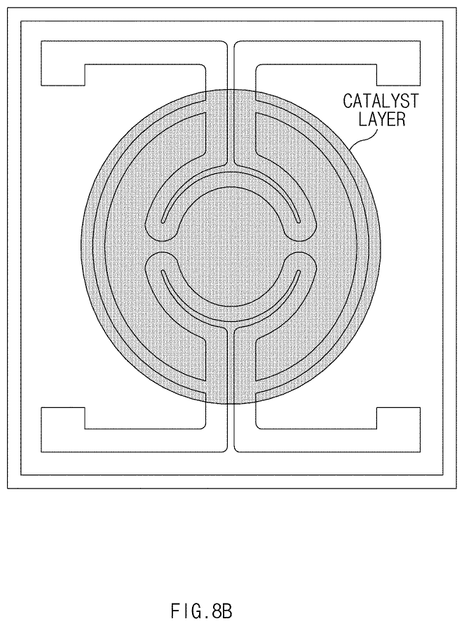

FIG. 8A illustrates a view for describing a circuit configuration for hydrogen measurement of a MEMS hydrogen sensor according to another embodiment of the present invention.

Referring to FIG. 8A, since the MEMS hydrogen sensor according to another exemplary embodiment of the present invention does not include a compensation element, a measurement circuit 500 and a temperature sensor 600 for temperature compensation may be used instead.

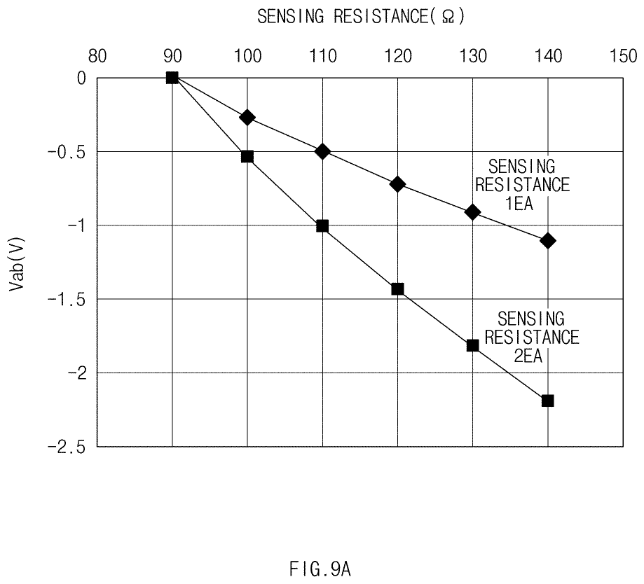

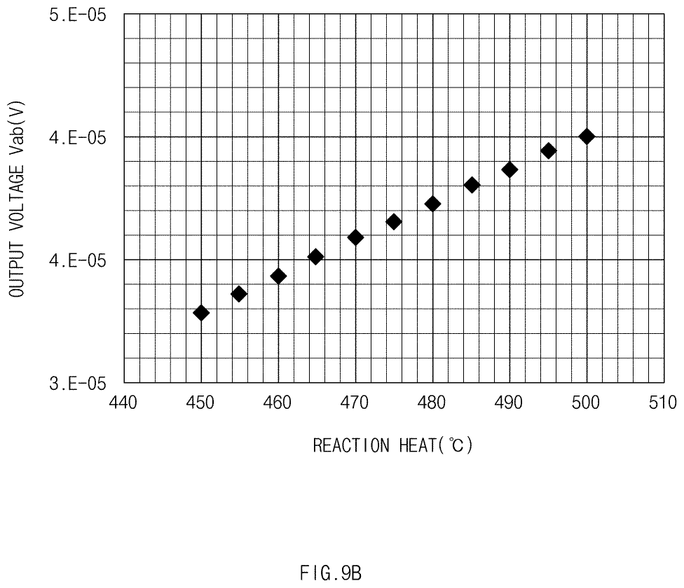

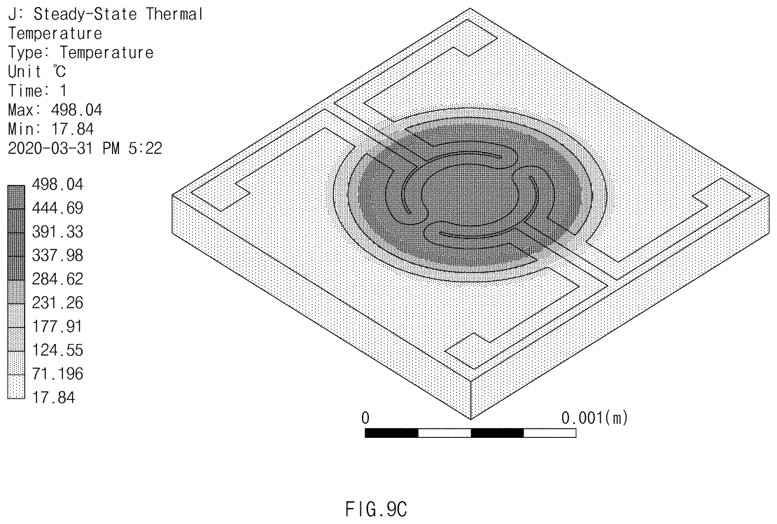

FIG. 8B illustrates a flowchart for describing a sensing area of a MEMS hydrogen sensor according to another exemplary embodiment of the present invention, and FIG. 9A to FIG. 9C illustrate views for describing an effect of a MEMS hydrogen sensor according to another exemplary embodiment of the present invention.

Referring to FIG. 8B, in the MEMS hydrogen sensor according to another exemplary embodiment of the present invention, the sensing area may increase as a number of the sensing elements R2 and R3 increases.

Referring to FIG. 9A, when the number of sensing elements is one or two, it indicates the change in the output voltage, and when the number of sensing elements is two as in the MEMS hydrogen sensor according to another exemplary embodiment of the present invention of FIG. 7, it can be seen that the change in the output voltage is larger compared to the case with one sensing element.

That is, in the case of two sensing elements, an output signal includes a resistance change value caused by an external temperature and a resistance change value caused by hydrogen. Accordingly, in order to compensate for the change in resistance caused by the external temperature, the measurement circuit 500 may measure an output signal of a hydrogen sensor in a sensor operating temperature environment, and may compensate the output signal by using a temperature sensing value measured by the temperature sensor 600.

That is, the measurement circuit 500 may map the resistance value of the sensing element for each temperature condition and then may output a value obtained by subtracting a value measured by the temperature sensor (e.g., resistance change caused by external temperature) from output values of the two sensing elements (resistance change value caused by hydrogen+resistance change value caused by external temperature) as a sensor output signal.

In FIG. 9B, it indicates that the output voltage increases as the reaction heat increases, and in FIG. 9C, it shows a temperature distribution due to the reaction heat. That is, as a result of analyzing the change in the output voltage Vab depending on an increase in a reaction heat caused by a hydrogen reaction in the catalyst layer, as the reaction heat increases, the resistance value of the sensing element may increase, and thus the hydrogen concentration may be predicted by monitoring a change in a linear output voltage.

As described above, embodiments of the present invention may reduce a cost and minimize the difference in resistance between elements through the configuration of the single element Wheatstone bridge circuit, and may manufacture the single element MEMS hydrogen sensor without increasing cost by increasing the sensing area by changing a pattern without additional processes.

The above description is merely illustrative of the technical idea of embodiments of the present invention, and those skilled in the art to which embodiments of the present invention pertains may make various modifications and variations without departing from the essential characteristics of the present invention.

Therefore, the exemplary embodiments disclosed in the present invention are not intended to limit the technical ideas of the present invention, but to explain them, and the scope of the technical ideas of the present invention is not limited by these exemplary embodiments. The protection range of the present invention should be interpreted by the claims below, and all technical ideas within the equivalent range should be interpreted as being included in the scope of the present invention.

What is claimed is:

1. A micro electro-mechanical systems (MEMS) hydrogen sensor comprising:
a sensing element configured to sense hydrogen gas;
an anti-icing element surrounding the sensing element; and
a compensation element configured to have a same resistance as that of the sensing element.

2. The MEMS hydrogen sensor of claim 1, further comprising a catalyst layer positioned on an upper portion of the sensing element and configured to react with the hydrogen gas.

3. The MEMS hydrogen sensor of claim 2, wherein the catalyst layer is platinum.

4. The MEMS hydrogen sensor of claim 1, wherein the sensing element is positioned in a center of the MEMS hydrogen sensor, and the compensation element is positioned in a first direction of the sensing element.

5. The MEMS hydrogen sensor of claim 4, wherein the anti-icing element includes:
a first anti-icing element positioned at opposite sides of the sensing element in a second direction crossing the first direction; and
a second anti-icing element.

6. The MEMS hydrogen sensor of claim 5, further comprising a plurality of electrode pads respectively positioned at ends of the sensing element, the compensation element, the first anti- icing element, and the second anti-icing element.

7. The MEMS hydrogen sensor of claim 5, wherein the MEMS hydrogen sensor is formed as a single element.

8. The MEMS hydrogen sensor of claim 7, wherein the sensing element, the compensation element, the first anti-icing element, and the second anti-icing element are four resistors of a Wheatstone bridge circuit.

9. A micro electro-mechanical systems (MEMS) hydrogen sensor comprising:
a first sensing element configured to sense hydrogen gas;
a second sensing element configured to sense the hydrogen gas; and
first and second anti-icing elements surrounding the first sensing element and the second sensing element.

10. The MEMS hydrogen sensor of claim 9, wherein:
the first anti-icing element is positioned at one side of the first and second sensing elements; and
the second anti-icing element is positioned at an opposite side of the first and second sensing elements.

11. The MEMS hydrogen sensor of claim 10, further comprising a catalyst layer positioned above the first and second sensing elements.

12. The MEMS hydrogen sensor of claim 11, wherein the catalyst layer is platinum.

13. The MEMS hydrogen sensor of claim 10, wherein:
resistance values of the first sensing element and the first anti-icing element are the same; and
resistance values of the second sensing element and the second anti-icing element are the same.

14. The MEMS hydrogen sensor of claim 13, wherein resistance values of the first and second anti-icing elements are configured to increase in response to hydrogen gas being sensed.

15. A micro electro-mechanical systems (MEMS) hydrogen sensing system comprising:
a MEMS hydrogen sensor comprising:
a first sensing element configured to sense hydrogen gas;
a second sensing element configured to sense the hydrogen gas; and
first and second anti-icing elements surrounding the first sensing element and the second sensing element;
a temperature sensor configured to sense an external temperature; and
a measurement circuit configured to compensate an output signal of the MEMS hydrogen sensor using a temperature sensing value from the temperature sensor.

16. The MEMS hydrogen sensing system of claim 15, wherein:
the first anti-icing element is positioned at one side of the first and second sensing elements; and
the second anti-icing element is positioned at an opposite side of the first and second sensing elements.

17. The MEMS hydrogen sensing system of claim 16, wherein the MEMS hydrogen sensor further comprises a catalyst layer positioned above the first and second sensing elements.

18. The MEMS hydrogen sensing system of claim 17, wherein the catalyst layer is platinum.

19. The MEMS hydrogen sensing system of claim 16, wherein:
resistance values of the first sensing element and the first anti-icing element are the same; and
resistance values of the second sensing element and the second anti-icing element are the same.

20. The MEMS hydrogen sensing system of claim 19, wherein resistance values of the first and second anti-icing elements are configured to increase in response to hydrogen gas being sensed.
